(19) United States
(12) Patent Application Publication
(10) Pub. No.: US 2023/0298878 A1
(43) Pub. Date: Sep. 21, 2023

(54) NANOTIP ION SOURCES AND METHODS

(71) Applicant: Brown University, Providence, RI (US)

(72) Inventors: Derek M. Stein, Providence, RI (US); Mathilde Lepoitevin, Paris (FR); Nicholas Drachman, Providence, RI (US)

(73) Assignee: Brown University, Providence, RI (US)

(21) Appl. No.: 17/917,672

(22) PCT Filed: Apr. 23, 2021
(86) PCT No.: PCT/US2021/028954

§ 371 Date: Oct. 7, 2022

Related U.S. Application Data
(60) Provisional application No. 63/015,407, filed on Apr. 24, 2020.

(51) Int. Cl. H01J 49/10; H01J 49/04; H01J 49/42; H01J 49/06; G01N 33/68

(52) U.S. Cl. CPC H01J 49/10; H01J 49/0404; H01J 49/4215; H01J 49/0445; H01J 49/067; H01J 49/061; G01N 33/6851

(57) ABSTRACT

The present disclosure generally relates in certain embodiments to the creation of ionized molecules, e.g., for detection in a mass spectrometer, or for other uses such as lithography, sputtering machines, propulsion etc. Some embodiments include an ion source comprising a capillary tip that may allow for direct ion evaporation of samples with an applied electric field. In some cases, the tip may have an opening with a cross-section less than 100 nm. In addition, certain aspects are directed to using a capillary tip that allow for detection of samples (e.g. amino acids), and in some cases allows for sequencing. For instance, some embodiments are directed to allowing single ions and ionic clusters to be evaporated at a high rate directly from aqueous samples in a mass spectrometer. Other aspects are directed to methods for making or using such ionized molecules, methods for making or using devices to create such ionized molecules, or the like.

### RELATED APPLICATIONS

This application claims the benefit of U.S. Provisional Patent Application Ser. No. 63/015,407, filed Apr. 24, 2020, entitled “Nanotip Ion Sources and Methods,” by Stein, et al., incorporated herein by reference in its entirety.

### FIELD

Certain aspects of the present disclosure are generally directed to the creation of ionized molecules.

### BACKGROUND

Advances in DNA sequencing techniques have made genomics studies extremely cheap and fast in recent years, but protein sequencing technology has not advanced at a similar pace. Electrospray ionization tandem mass spectrometry (ESI-MS/MS) is a common tool used for protein sequencing, but the technique faces a number of challenges that a single molecule method could address. For example, the electrospray ion source emits ionized peptide fragments contained in large, multiply charged droplets. Generally, a background gas is introduced into the path of the droplets to dry them out, initiating a series of Coulomb fission events, eventually yielding individual ions. This necessary but chaotic drying process spreads ions into a plume, destroying the original sequence and requiring complex algorithms to identify the peptide fragments and reconstruct the amino acid sequence. Due to the chaotic process by which the electrosprayed droplets break down into ever-smaller droplets, eventually yielding singly charged ions via ion evaporation, only a small fraction of the sample molecules makes it to the mass analyzer.

Ion evaporation has previously been induced from molten metals, ionic liquids, and high concentration solutions of salt dissolved in glycerol. It is generally understood that a high liquid conductivity and low flow rate facilitate ion evaporation. Previous attempts to induce ions to evaporate from aqueous solutions encountered significant problems related to the high flow rates or the low conductivities of the liquid, as well as freezing and electrical arcing. Poor mass spectra were consequently obtained while large amounts of sample were consumed. Volatile liquids, like water, further complicate ion evaporation because the evaporation of solvent molecules from the liquid meniscus into high vacuum cools the liquid, and in the case of aqueous solutions, the evaporation of water can lead to freezing of the liquid. As a result of these issues, ion evaporation from aqueous solutions has remained out of reach.

### SUMMARY

Certain aspects of the present disclosure are generally directed to the creation of ionized molecules. The subject matter of the present disclosure involves, in some cases, interrelated products, alternative solutions to a particular problem, and/or a plurality of different uses of one or more systems and/or articles.

One aspect of the present disclosure is generally directed to an ion source. According to one set of embodiments, the ion source comprises a capillary defining an opening having a cross-section of less than 125 nm or 100 nm; and an electrode positioned proximate the opening of the capillary in a downstream direction.

According to another set of embodiments, the ion source comprises a capillary defining an opening having a cross-section of less than 125 nm or 100 nm; and an electrode positioned proximate the opening of the capillary in a downstream direction.

The ion source, in yet another set of embodiments, comprises a capillary defining an opening; and an electrode positioned to create an electric field having an electric field maximum proximate the opening of the capillary, wherein the opening of the capillary is sized such that, when the electric field is applied, a fluid within the capillary forms a charged meniscus and species exit the charged meniscus, wherein at least 50% of the exiting species exit the charged meniscus via ion evaporation.

Another aspect is generally directed to a method. In one set of embodiments, the method comprises passing a fluid into a capillary defining an opening; and applying an electric field at least sufficient to cause molecules within the fluid to exit the fluid, wherein the opening is sized to cause at least 50% of the molecules to exit as ions or ion clusters.

The method, in another set of embodiments, comprises passing a fluid into a capillary defining an opening having a cross-section of less than 125 nm or less than 100 nm; applying an electric field to ionize molecules proximate the opening to produce ions or ion clusters; and passing the ions or ion clusters from the fluid directly into an environment having a pressure of no more than 100 mPa.

Yet another aspect is generally directed to a method of sequencing a biopolymer. In one set of embodiments, the method comprises passing a fluid comprising a biopolymer into a capillary defining an opening having a cross-section of less than 125 nm; applying an electric field to ionize the biopolymer proximate the opening to produce ions or ion clusters; directing the ions or ion clusters to a detector; and determining a sequence of the biopolymer by determining the ions or ion clusters with the detector.

The method of sequencing a biopolymer, in another set of embodiments, comprises passing a fluid comprising a biopolymer into a capillary defining an opening; applying an electric field to ionize the biopolymer proximate the opening to produce ions or ion clusters; passing the ions or ion clusters directly into an environment having a pressure of no more than 100 mPa; directing the ions or ion clusters to a detector; and determining a sequence of the biopolymer by determining the ions or ion clusters with the detector.

Still another aspect is generally directed to a nanopore mass spectrometer. According to one set of embodiments, the nanopore mass spectrometer comprises an ion source comprising a capillary and an electrode proximate the capillary, wherein the capillary comprises an opening having a cross-section of less than 125 nm; a vacuum chamber housing the ion source; ion optics downstream of the ion source; a mass filter downstream of the ion optics; and a detector further downstream of the mass filter.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Various aspects and embodiments of the application will be described with reference to the following figures. It should be appreciated that the figures are not necessarily drawn to scale.

FIGS. 1A-1B show a nanopore mass spectrometer, in accordance with one embodiment. FIG. 1A shows a schematic of the nanopore mass spectrometer instrument.

FIG. 1B is an SEM image of a quartz capillary with an inner tip diameter of 30 nm.

FIG. 2 shows a mass spectrum of arginine obtained with nanopore mass spectrometry, in accordance with another embodiment of the disclosure.

FIG. 3 shows arginine spectra, in accordance with some embodiments of the disclosure.

FIG. 4 shows a gallery of amino acid mass spectra, in accordance with some embodiments of the disclosure described herein.

FIGS. 5A-5B show ion evaporation with nanoscale tips, in accordance with some embodiments of the disclosure. FIG. 5A shows an electric field used in ion evaporation. FIGS. 5B and 5C show a comparison of an electrospray ion source used in conventional ESI experiments (FIG. 5B) and a nanopore ion source (FIG. 5C).

FIGS. 6A-6B show cross-sectional views of components in an ion source, in accordance with certain embodiments. FIG. 6A shows a capillary defining an opening. FIG. 6B shows a capillary defining an opening and an electrode proximate the opening of the capillary.

FIGS. 7A-7C show cross-sectional views of components of an ion source, in accordance with certain embodiments. FIG. 7A shows a stationary fluid within the capillary. FIG. 7B shows charging of a fluid within the capillary under an applied electric field. FIG. 7C shows a fluid within the capillary under an electric field generated by an electrode proximate the capillary.

FIG. 8 shows a cross-sectional view of a mass spectrometer comprising an ion source in accordance with certain embodiments.

### DETAILED DESCRIPTION

The present disclosure generally relates in certain embodiments to the creation of ionized molecules, e.g., for detection in a mass spectrometer, or for other uses such as lithography, sputtering machines, propulsion, etc. Some embodiments include an ion source comprising a capillary tip that may allow for direct ion evaporation of samples with an applied electric field. In some cases, the tip may have an opening with a cross-sectional dimension (e.g., diameter) less than 125 nm or 100 nm, etc. In addition, certain aspects are directed to using a capillary tip that allow for detection of samples (e.g., amino acids), and in some cases allows for sequencing. For instance, some embodiments are directed to allowing single ions and ionic clusters to be evaporated at a high rate directly from aqueous samples in a mass spectrometer. Other aspects are directed to methods for making or using such ionized molecules, methods for making or using devices to create such ionized molecules, or the like.

For example, some embodiments are generally directed to an ion source comprising a capillary and an electrode, which may be annular in some cases, between which a voltage is applied to produce ions. In some cases, the capillary may have an inner tip diameter of less than 125 nm or 100 nm, etc. This may allow ions to evaporate directly off of the meniscus of a fluid in the capillary, bypassing the wasteful droplet evaporation process. In this regime, ion evaporation may account for the majority of the ionic current, and this emission mode can be achieved in some cases with relatively low salinity solutions. In some embodiments, tips with inner diameters less than 125 nm or 100 nm, etc. may be able to produce a high fraction of bare ions or ionic clusters, for example, comprising small numbers of solvent molecules, e.g., only 1 or 2 solvent molecules. The small area of the liquid vacuum interface may in some cases prevent significant evaporative heat loss, which allows the use of volatile solvents like water in certain cases. Methods such as these could be used in some embodiments to analyze molecules or ions, e.g., biomolecules such as amino acids, nucleic acids, peptides or proteins, etc. In some cases, ion sources such as those described herein may improve the sensitivity of mass spectrometry experiments, allow single-molecule protein sequencing or single cell proteomic studies. Other applications such as those described below are also possible.

For example, some embodiments are generally directed to an ion source comprising a capillary and an electrode. The electrode may be used to generate ionized molecules directly from a fluid within the capillary, e.g., into a reduced pressure environment or vacuum, e.g., at a pressure of 100 mPa, or other pressures described herein. In certain embodiments, the opening of the capillary is sized such that, when an electric field is applied, a fluid within the capillary forms a charged meniscus and species within the fluid exit the charged meniscus, e.g., via predominately ion evaporation. The use of capillaries with a submicron opening (e.g., less than 125 nm or 100 nm, etc.) may favor the ionization of a fluid via ion evaporation, where the species exiting the capillary directly ionizes into single charged ions or charged ion clusters, in contrast to electrospray ionization, where the species exiting the capillary exit via a liquid jet that breaks up into charged droplets that further break down into charged ions in the presence of a background gas, although it should be understood that some electrospray ionization may still occur in some cases. Ion evaporation may be preferred in certain applications, e.g., that require the efficient use or generation of single ions from a fluid. For example, certain embodiments are related to ion sources in mass spectroscopy, where single charged ions can be directly generated and subsequently detected.

According to one set of embodiments, the ion source comprises a capillary defining an opening having a cross-sectional dimension (e.g., inner diameter of the capillary) of less than 100 nm. The opening may also be sized in some cases such that when an electric field is applied, ion evaporation dominates over liquid jet formation. For instance, in certain embodiments, at least 50% of the exiting species may exit via ion evaporation or in the form of ions or ion clusters. For instance, a nanoscale capillary can allow ions to evaporate directly off of a fluid meniscus. In some embodiments, a fluid can be passed into a capillary having such an opening, and directly delivered into a reduced pressure or vacuum environment (e.g., having a pressure of no more than 100 mPa) in the form of ions and ion clusters. The ions and ion clusters can be analyzed by a mass filter and an ion detector in a mass spectrometer, or applied to other applications such as those described herein.

In addition, certain aspects are related to methods of sequencing molecules or polymers, such as biopolymers, from a fluid within the capillary (e.g., via mass spectrometry). In some embodiments, the fluid comprises a polymer, such as a biopolymer, dissolved in a solvent having relatively high vapor pressure (e.g., water). Typically, the evaporation of a high volatility solvent (e.g., water) can lead to freezing of the fluid at the opening of the capillary as the solvent evaporates, thus limiting the mass spectrometer's ability to successfully evaporate the ions from the fluid. However, the opening of the capillary may be sized such that the fluid meniscus at the opening, e.g., as discussed herein, may have a smaller area that may reduce these effects. Thus, the use of capillary with small openings in the ion source of a mass spectrometer can allow the study of molecules, for example, polymers or biopolymers such as amino acids, nucleic acids, and peptides or proteins, in aqueous solutions. Molecules that are not polymers may also be studied in certain embodiments.

To sequence a molecule such as a polymer (e.g., a biopolymer), certain embodiments are directed to applying an electric field to ionize the molecule proximate the opening of a capillary to produce ionized fragments. In certain embodiments, the ionized fragments from the fluid are directly passed into a reduced pressure environment. The ionized fragments in some cases may comprise single ions or ion clusters, as discussed herein, for instance, having a small number of solvent molecules (e.g., water). The opening may be sized such that ionized fragments exit the opening in a sequential order according to the sequence of the molecule. For instance, certain embodiments allow for the determination of a sequence of the molecule by determining ionized fragments produced by ionizing the molecule within the detector.

In addition, certain aspects are directed to devices comprising an ion source having a capillary as disclosed herein. The device may also have an electrode proximate the capillary. It should be noted that although some embodiments disclose the use of the ion source in a mass spectrometer, the use of the ion source as disclosed herein does not apply solely to a mass spectrometer. The ion source could also be used, for example, in lithography machines, sputtering machines, space propulsion systems, etc., as discussed herein.

Certain aspects are directed to an ion source comprising a capillary defining an opening and an electrode posited proximate the opening. The capillary may have an opening at an end or a tip of the capillary. The opening may have any of a variety of cross-sectional dimensions, and may be of any shape, e.g., circular, elliptical, square, etc. In some embodiments, the opening comprises a cross-sectional dimension of less than 150 nm, less than 130 nm, 125 nm, less than 120 nm, less than 110 nm, less than 100 nm, less than 90 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, less than 10 nm, less than 5 nm, less than 2 nm, etc. In addition, the opening, in some cases, may have a cross-sectional dimension of at least 1 nm, at least 5 nm, at least 10 nm, at least 15 nm, at least 20 nm, at least 25 nm, at least 30 nm, at least 40 nm, at least 50 nm, at least 60 nm, at least 70 nm, at least 80 nm, at least 90 nm, etc. Combinations of these are also possible; for example, the opening may have a cross-sectional dimension of between 50 nm and 100 nm. While the above embodiment describes a capillary having an opening at the end or the tip of the capillary, it should be understood that not all embodiments described herein are so limiting, and in certain embodiments, the capillary may additionally or alternatively have a plurality of openings along the side of the capillary. In addition, in some cases, a device may have one or more apertures or openings, e.g., in a channel or other structure. Thus, an opening need not be the opening of a capillary.

In some embodiments, the capillary is tapered at the opening. For instance, the capillary may have a constant tapering, e.g., such that the tip of the capillary is cone-shaped. Any suitable angle may be present. For example, the angle may be less than 15 degrees, less than 10 degrees, less than 9 degrees, less than 8 degrees, less than 7 degrees, less than 6 degrees, less than 5 degrees, less than 4 degrees, less than 3 degrees, less than 2 degrees, or less than 1 degree (where 0 degrees indicates no taper, i.e., the capillary is cylindrical. In addition, in some cases, the angle of the taper may be at least 1 degree, at least 3 degrees, at least 5 degrees, etc., in certain cases. Combinations of these ranges are also possible, e.g., the tapering may be between 1 degree and 5 degrees.

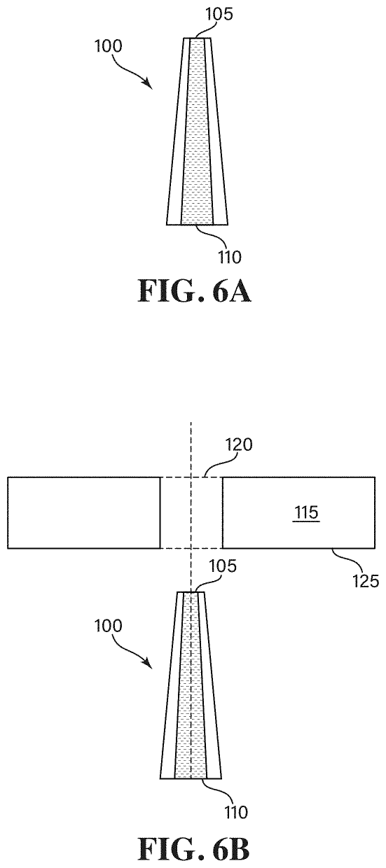

For instance, one example of an embodiment of a capillary is now shown in FIG. 6A in a cross-sectional view. In this example, the capillary 100 has an opening 105, where a cross-section 110 of the capillary 100 tapers down gradually to the opening 105. In this example, the opening comprises a cross-sectional dimension (e.g., inner diameter) of less than 125 nm or less than 100 nm etc.

In certain embodiments where the capillary is tapered at the opening, a laser pulling technique can be used to fabricate the tapered opening. It should be understood that techniques other than a laser-pulling technique could also be used to produce capillaries with tapered openings. It should also be understood that, although the capillary discusses herein has a tapered opening, in other examples, the opening of the capillary could be non-tapered.

The capillary of the ion source comprises quartz in certain embodiments. Additional examples of materials that can be used to fabricate the capillary include, but are not limited to, glass (e.g., borosilicate glass), a plastic, a metal, a ceramic, a semiconductor, a carbon nanotube, a boron nitride nanotube, etc.

In some embodiments, the capillary has a relatively high aspect ratio, e.g., a ratio of the length of the capillary to the cross-sectional dimension (e.g., diameter) of the capillary's opening. For example, the capillary may have an aspect ratio that is greater than 10,000. However, it should be understood that the aspect ratio is not so limited. For instance, in some examples, the aspect ratio of the capillary length to the opening's cross-sectional dimension may be greater than 10, greater than 100, greater than 1,000, greater than 10,000, greater than 100,000, or greater than 1,000,000.

The capillary may have a circular or a non-circular cross-section (e.g., square). In addition, in some embodiments, the capillary may have a relatively small cross-section, e.g., diameter. For instance, the cross-sectional dimension of the capillary may be less 200 nm, less than 150 nm, less than 75 nm, less than 60 nm, less than 50 nm.

Certain embodiments of the ion source also comprise an electrode positioned proximate the capillary, e.g., the opening of the capillary. The electrode may be used to apply an electric field (for example, as described below) to a fluid within the capillary, e.g., to be applied to the meniscus. In some cases, the fluid within the capillary may be in contact with a counterelectrode, e.g., such that a voltage difference between the electrode proximate the opening of the capillary and the counterelectrode within the capillary is able to generate an electric field to the fluid. In some embodiments, the electrode may be positioned so as to cause an electric field maximum proximate the opening of the capillary. For example, in some embodiments, the electrode may be positioned within 50 mm, within 40 mm, within 30 mm, within 20 mm, within 15 mm, within 10 mm, within 5 mm, within 3 mm, within 2 mm, within 1 mm, etc., of the opening of the capillary.

The electrode, in some embodiments, may be positioned around the capillary, or may be positioned in front of the capillary, e.g., in front of the opening of the capillary, or in a downstream direction.

The electrode may have any suitable shape. In some cases, the electrode is circular or circularly symmetric, or is symmetrically positioned with respect to the capillary. However, other shapes or arrangements are also possible.

In some embodiments, the electrode defines an opening (e.g., an aperture). Thus, the electrode may be annular in some cases. The electrode may be positioned such that ions or ion clusters escaping the fluid in the capillary pass through the center opening of the electrode. The center opening of the electrode can be of any shape, including, but not limited to, a circular shape that can be positioned annularly around the opening of the capillary. The opening may also be non-circular in some cases. In some embodiments, the opening of the electrode is positioned coaxially to the opening of the capillary. That is, the opening can be aligned, in certain embodiments, to the opening of the capillary, e.g., such that an imaginary line passing through the center of a cross-section of the capillary passes through the center opening of the electrode. This may facilitate the application of an electric field to the fluid in the capillary, e.g., to cause ions or ion clusters to exit the fluid, as discussed herein.

For example, in some embodiments, the electrode has a center opening with cross-sectional dimension (e.g., inner diameter) greater than the cross-sectional dimension of the opening of the capillary, e.g., at the end or tip of the capillary. For instance, in accordance to certain embodiments, the electrode has a center opening with a cross-sectional dimension (e.g., inner diameter) at least 5 times greater than the cross-sectional dimension of a capillary's opening. However, it should be understood that the ratio of the cross-sectional dimensions of the electrode's center opening to the capillary's opening is not limited. For instance, in some examples, the cross-sectional dimension of the center opening of the electrode could be at least 2 times, at least 3 times, at least 5 times, at least 10 times, at least 20 times, at least 30 times, at least 50 times, at least 75 times or at least 100 times larger than the cross-sectional dimension of the capillary's opening. In certain cases, the opening of the electrode may have a cross-sectional dimension of less than 10 cm, less than 5 cm, less than 3 cm, less than 1 cm, less than 5 mm, less than 3 mm, less than 1 mm, etc. In addition, in some embodiments, a front side of the electrode is positioned in front of the opening of the capillary.

For instance, one example of an embodiment of an electrode is now shown in FIG. 6B in a cross-sectional view. In this example, an electrode 115 having a front side 125 is positioned in front of the opening 105 of the capillary 100 in a way such that the axis of the electrode 115 is aligned with the axis of the capillary 100.

In addition, the electrode itself can be of any shape (e.g., circular or non-circular). The electrode may have the same or a different shape than its opening (if present). The electrode may have any suitable cross-sectional dimension. For example, the electrode may have a cross-sectional dimension of less than 10 cm, less than 5 cm, less than 3 cm, less than 1 cm, less than 5 mm, less than 3 mm, less than 1 mm, etc.

In some embodiments, the electrode comprises steel. Other examples include copper, graphite, silver, aluminum, gold, electrically conducting ceramics, or the like.

Thus, certain embodiments are directed to an electrode able to generate an electric field. In some cases, as noted, the electrode may be positioned to create an electric field maximum proximate the opening of the capillary. In some embodiments, a fluid is housed in the capillary such that when an electric field is applied by the electrode proximate the opening of the capillary, molecules within the fluid can ionize and exit from the opening of the capillary, e.g., as ions or ion clusters such as discussed herein. In some cases, for example, the electrode and the capillary (e.g., the interior of the capillary) may be connectable to a voltage source, e.g., as discussed herein.

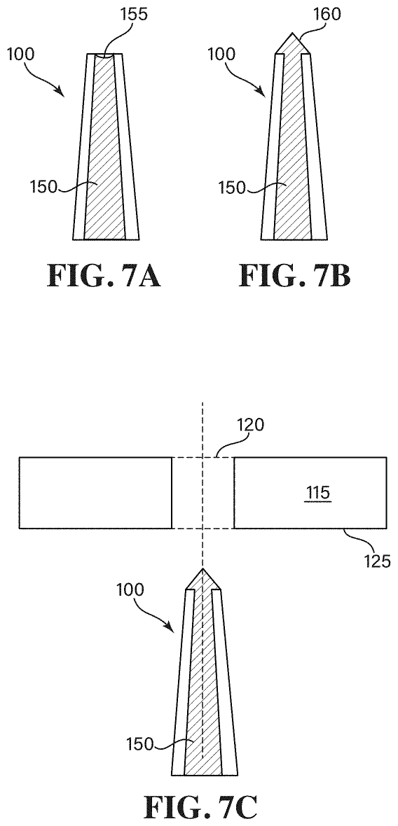

One non-limiting example of an embodiment is shown in FIGS. 7A-7C in cross-sectional views. In this example, before the application of an electric field, a fluid 150 remains stationary inside the capillary and has a fluid meniscus 155, as shown in FIG. 7A. As an electric field having an electric field maximum proximate the opening of the capillary is applied, the fluid 150 within the capillary 100 forms a charged fluid meniscus in the shape of a cone 160 (e.g., a Taylor cone) as a result of charge-charge repulsion within the fluid 150 induced by the electric field, as illustrated in FIG. 7B. As illustrated in FIG. 7C, the fluid 150 subsequently ionizes from the conical meniscus under the influence of an electric field and exit from the opening 105 of the capillary through the center opening 120 of the electrode 115.

Thus, in certain embodiments, the voltage source, in conjunction with the electrodes, may be used to produce an electric field to cause ions or ion clusters to exit a fluid in the capillary, e.g., as discussed herein. In some embodiments, a voltage is applied to generate an electric field at least sufficient to ionize molecules within the fluid at the opening of the capillary, e.g., to produce ions or ion clusters. For instances, in certain embodiments, a voltage in the range of 80 V to 400 V could be used to generate an electric field. In some cases, the voltage may be at least 40 V, at least 60 V, at least 80 V, at least 100 V, at least 120 V, at least 140 V, at least 160 V, at least 180 V, at least 200 V, at least 220 V, at least 240 V, at least 260 V, at least 280 V, at least 300 V, at least 320 V, at least 340 V, at least 360 V, at least 380 V, at least 400 V, at least 450 V, at least 500 V, at least 600 V, etc. In addition, in some cases, the voltage may be no more than 600 V, no more than 500 V, no more than 450 V, no more than 400 V, no more than 380 V, no more than 360 V, no more than 340 V, no more than 320 V, no more than 300 V, no more than 280 V, no more than 260 V, no more than 240 V, no more than 220 V, no more than 200 V, no more than 180 V, no more than 160 V, no more than 140 V, no more than 120 V, no more than 100 V, no more than 80 V, no more than 60 V, etc. In some cases, combinations of these voltages are possible. For instance, the voltage may be applied between 80 V and 360 V, etc. The voltage may be applied as a constant voltage, or a varying or periodic voltage in certain cases.

As mentioned, a voltage may be applied to create an electric field maximum proximate the opening of the capillary, or the fluid within the capillary (e.g., at the meniscus at the opening). For example, a voltage may be applied to create an electric field maximum of at least 0.5 V/nm, at least 0.7 V/nm, at least 1 V/nm, at least 1.1 V/nm, at least 1.3 V/nm, at least 1.5 V/nm, at least 2 V/nm, at least 2.5 V/nm, at least 3 V/nm, at least 3.5 V/nm, at least 4 V/nm, etc. In certain embodiments, the electric field maximum may be no more than 5 V/nm, no more than 4.5 V/nm, no more than 4 V/nm, no more than 3.5 V/nm, no more than 3 V/nm, no more than 2.5 V/nm, no more than 2 V/nm, no more than 1.5 V/nm, no more than 1 V/nm. Combinations of these ranges are also possible in some embodiments; for example, the electric field may be between 1.5 V/nm and 3.0 V/nm, between 1.5 V/nm and 4.0 V/nm, etc.

Without wishing to be bound by any theory, it is believed that in certain embodiments, when the electric field is applied, fluid within the capillary forms a charged meniscus and species exit the charged meniscus, e.g., as ions or ion clusters. In some cases, the opening of the capillary may be sized such that at least 10% of the exiting species exit via ion evaporation, e.g., as ions or ion clusters. In some cases, at least 20%, at least 30%, at least 40%, at least 50%, at least 60%, at least 70%, at least 80%, at least 90%, at least 95%, etc. of the exiting species exit via ion evaporation.

As described previously, according to certain embodiments, a charged fluid meniscus in the shape of a cone can be generated at the opening of a capillary under an electric field. In some embodiments, the conical fluid meniscus acts as a point source to allow species to exit as ions or ion clusters.

The fluid meniscus could produce exiting species via mechanisms such as charged droplets via electrospray ionization, and/or ions and ion clusters via ion evaporation. However, in the case of electrospray ionization, the exiting species exiting from the liquid meniscus would exit as charged droplets of fluid containing the exiting species, which would require the presence of a background gas to further break down the droplets into individual ions, typically via a Coulomb fission process. Ion evaporation, on the other hand, describes a process where a molecule is directly ionized into ions (e.g., bare ions) or ion clusters (e.g., ions with solvent molecules), instead of charged droplets. An ion cluster may contain a single ion and a plurality of solvent molecules, usually a relatively small number. For example, the ion clusters may contain no more than 10, no more than 9, no more than 8, no more than 7, no more than 6, no more than 5, no more than 4, no more than 3, no more than 2, or no more than 1 solvent molecule.

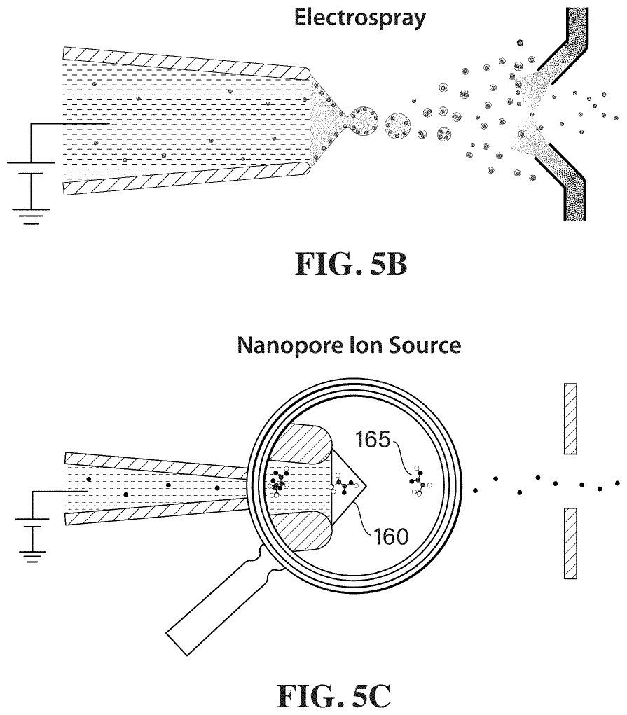

Thus, for example, in some embodiments, the opening of the capillary is sized (e.g., the cross-sectional dimension of the opening is less than 125 nm or 100 nm, etc.) such that the formation of charged droplets can be avoided, and such that at least 50% of the exiting species directly ionize as ions or ion clusters from conical fluid meniscus at the opening of the capillary. For instance, one such example is now shown in FIG. 5C. In this example, exiting species 165 can be seen exiting from the conical fluid meniscus 160 at the opening of a capillary under an applied electric field. The opening of the capillary is sized such that the conical fluid meniscus allows the exiting species 165 to exit in the form of ions or ion clusters, as shown in FIG. 5C.

As mentioned, in some embodiments, a capillary having a relatively small opening (e.g., a cross-sectional dimension of less than 125 nm or 100 nm, etc.) may be associated with the production of a relatively small number of solvent molecules in an ion cluster, e.g., as described above. In some embodiments, the opening of the capillary may be sized (e.g., less than 125 nm or 100 nm, etc.) such that the plurality of solvent molecules comprises less than or equal a certain number of solvent molecules, e.g., such that on average, the ion clusters produced by the ion source contain less than or equal to 7, 6, 5, 4, 3, or 2 solvent molecules. In some embodiments, a substantial number (e.g., greater than or equal to 50%, greater than or equal to 60%, greater than or equal to 70%, greater than or equal to 80%, greater than or equal to 85%, greater than or equal to 90%, greater than or equal to 95%, greater than or equal to 99%, or all) of the ion clusters contains one or two solvent molecules.

In addition, as discussed, certain aspects are directed to methods of ionizing a fluid using an ion source, e.g., to produce single ions or ion clusters. Certain embodiments comprise passing a fluid into a capillary defining an opening having a cross-sectional dimension less than 125 or 100 nm, etc., or other configurations such as those discussed herein.

In some embodiments, the fluid comprises a sample and a solvent. The sample may include any species of interest that can be ionized from the opening of the capillary. For instance, in accordance with certain embodiments, a species of interest comprises a biopolymer (e.g., nucleic acids such as DNA or RNA, peptides or proteins, etc.). Other examples include other types of polymers, e.g., nylon, polyethylene, etc., or other species of interest that are not necessarily polymers, e.g., biomolecules. Non-limiting examples of biomolecules may include monomers such as amino acids, nucleotides, etc. In some cases, the species of interest is unknown, and it is desired that the structure of the species be at least partially determined, e.g., by ionizing the species and detecting the ion fragments, such as in mass spectroscopy or other related techniques.

In some embodiment, the solvent may be any liquid that can be used to dissolve the sample or the species of interest. For instance, in accordance with certain embodiments, the solvent comprises water. However, the solvent is not limited to water. In some cases, the solvent may be an aqueous solution, e.g., having any of a variety of salt concentrations. In some embodiments, an aqueous solution may have a salt concentration of greater than or equal to 10 mM, greater than or equal to 20 mM, greater than or equal to 30 mM, greater than or equal to 50 mM, greater than or equal to 100 mM, greater than or equal to 150 mM, greater than or equal to 200 mM, greater than or equal to 300 mM, greater than or equal to 400 mM, greater than or equal to 500 mM, greater than or equal to 750 mM, greater than or equal to 1 M, greater than or equal to 2 M, greater than or equal to 5 M, or greater than or equal to 7.5 M. In some embodiments, an aqueous solution may have a salt concentration of less than or equal to 10 M, less than or equal to 7.5 M, less than or equal to 5 M, less than or equal to 2 M, less than or equal to 1 M, less than or equal to 750 mM, less than or equal to 500 mM, less than or equal to 400 mM, less than or equal to 200 mM, less than or equal to 150 mM, less than or equal to 100 mM, less than or equal to 50 mM, less than or equal to 30 mM, less than or equal to 20 mM, less than or equal to 10 mM, etc. Combination of the above-referenced ranges are possible (e.g., greater than or equal to 100 mM and less than or equal to 10 M, or greater than or equal to 150 mM and less than or equal to 1 M).

Additional examples of solvents that can be used include, but are not limited to, formamide, alcohols (e.g., ethanol, isopropanol, etc.), organic solvents (e.g., toluene, acetonitrile, acetone, hexane, etc.), ionic liquids, inorganic solvents (e.g., ammonia, sulfuryl chloride fluoride, liquid acids and bases, etc.). Combinations of any of these and/or other solvents are also possible in certain cases.

In addition, in some embodiments, the fluid comprises a solvent (e.g., water) having a relatively high volatility, e.g., to facilitate the production of ions or ion clusters. For instance, water, with its boiling point of 100° C., can be considered to be volatile in some cases. In some embodiments, liquids with boiling points close to room temperature could be used to facilitate the production of ions or ion clusters. In some embodiments, a solvent that could be used to facilitate the production of ions or ion clusters may have a boiling point of less than or equal to 100° C., less than or equal to 80° C., less than or equal to 60° C., less than or equal to 40° C., less than or equal 20° C., etc. In addition, the solvent may have a boiling point greater than or equal to 10° C., greater than or equal to 30° C., greater than or equal to 50° C., greater than or equal to 70° C., greater than or equal to 90° C., etc. Combination of these are also possible; for example, the solvent may have a boiling point of between 50° C. and 100° C. Additional examples of solvents having a relatively high volatility include, but are not limited to, acetone, isopropanol, hexane, etc.

In some embodiments, the temperature of the capillary (in addition to the type of fluid it contains) may be varied to control the number of solvent molecules in a resultant ion cluster. In some embodiments, the temperature of the capillary is set such that the plurality of solvent molecules comprises less than or equal a certain number of solvent molecules, e.g., such that on average, the ion clusters produced by the ion source contain less than or equal to 7, 6, 5, 4, 3, or 2 solvent molecules. In some embodiments, the temperature is at least 20° C., at least 30° C., at least 40° C., at least 50° C., at least 60° C., or at least 70° C. In some embodiments, the temperature is no more than 80° C., no more than 70° C., no more than 60° C., no more than 50° C., no more than 40° C., no more than 30° C. Combination of the above-referenced ranges are possible (e.g., greater than or equal to 20° C. and less than or equal to 80° C.). In some cases, the temperature of the capillary is controlled by a resistive heater, by a Peltier junction, by an infrared heater, etc.

In some embodiments, an appropriate range of electric field and an appropriate range of sizes of the capillary opening can be selected to cause at least some of the molecules to exit as ions or ion clusters, e.g., as discussed herein.

Certain embodiments comprise passing the ionized molecules from the fluid directly into a reduced pressure or vacuum environment. Without wishing to be bound by any theory, it is noted that techniques such as electrospray ionization typically requires the presence of a background gas to further break down the droplets into individual ions, typically via a Coulomb fission process. In contrast, in accordance with certain embodiments, ions or ion clusters produced as discussed herein can be directly passed into such an environment, without requiring substantial amounts of background gas. Thus, certain techniques such as mass spectrometry may be performed using a reduced pressure or vacuum environment, without necessarily requiring the addition of a background gas.

Thus, in one set of embodiments, the capillary may be positioned to allow ions or ion clusters exiting the opening to enter a reduce pressure or vacuum environment. In some cases, the environment may be an environment having a pressure of no more than 100 mPa. In certain embodiments, the environment may have a pressure of no more than 1000 mPa, no more than 10 mPa, no more than 1 mPa, no more than 0.1 mPa, etc. In some embodiments, the ions or ion clusters from the fluid are passed directly into a vacuum environment.

It should be understood that some of the embodiments provided herein focused on passing the ionized molecules from the fluid directly into an environment having a pressure of no more than 100 mPa. However, it should be understood the pressure within the environment is not limited to 100 mPa. In some embodiments, the pressure could also be greater than or equal to 100 mPa, and less than or equal to 1 Pa.

In some embodiments, the mass spectrometer comprises a pump. The pump may be used to create a reduced pressure or vacuum environment, e.g., as discussed herein. Non-limiting examples of pumps include diffusion pumps, molecular drag pumps, turbomolecular pumps, or the like.

In some embodiments, there may be a relatively high pressure difference between the vacuum chamber and the fluid at the capillary opening. For instance, the pressure may be about 1 atmosphere at where the fluid enters in the capillary and about 100 mPa, or other reduced pressures such as those described herein, inside the vacuum chamber where the opening of the capillary is located. However, in some cases such as are described herein, the fluid meniscus at the opening of the capillary may be relatively stable despite the relatively high pressure difference, e.g., due to the surface tension of the fluid at the meniscus. For instance, the pressure difference across the fluid meniscus at the opening of the capillary may be at least 0.1 atm, at least 0.2 atm, at least 0.3 atm, at least 0.4 atm, at least 0.5 atm, at least 0.6 atm, at least 0.7 atm, at least 0.8 atm, at least 0.9 atm, at least 1 atm, etc. Furthermore, in some embodiments, the hydraulic resistance of a fluid in a capillary such as described herein (e.g., a capillary with an opening less than 100 nm) may be higher than that in an ion source employed in electrospray ionization.

In accordance with certain embodiments, the opening of the capillary is sized such that a solvent having a relatively high volatility remain unfrozen at the opening of the capillary when exposed to relatively low pressures. In some embodiments, the opening of the capillary is small enough such that a solvent of relatively high volatility remains unfrozen as the solvent enters the surrounding environment. In some embodiments, the opening of the capillary is small enough such that a fluid comprising a sample and a solvent remains unfrozen as the species of interest ionizes, such that at least some of the species of interest ionizes to form ions (e.g., single ions) or ion clusters.

Some aspects are directed to a mass spectrometer comprising an ion source as described herein. However, ion sources such as described herein are not limited to only mass spectrometers, but can be used in other applications, such as lithography, sputtering machines, propulsion (e.g., space propulsion), etc. As a non-limiting example, in lithography, focused ion beam (FIB) machines can be used to examine and/or modify lithography masks, and/or to etch features into materials by sputtering. Sputtering is a process by which atoms are removed from the surface of a solid by ions that impinge with high kinetic energy. In some embodiments, the ion source described herein is present in a focused ion beam (FIB) machine, which may be used to deliver molecules to a substrate material in a patterned fashion.

In certain embodiments, an ion source as described herein may be used with a liquid-chromatography mass spectrometry system. For instance, a liquid chromatograph can be coupled with an ion source to separate peptides or other molecules before ionizing and delivering them into a mass spectrometer. In some cases, the mass spectrometer may be used to perform a single or tandem (MS/MS) analysis to identify the ionized peptides or molecules, as in a proteomics experiment. Advantageously, the use of the ion source (having a capillary with a nanosized opening and/or tip) described herein to deliver ions directly into a low pressure environment may improve the sensitivity of the instrument, the ion transmission efficiency in such a system, and remove the need for multiple pumping stages.

In certain embodiments, an ion source as described herein may be used as both a nanopipette and an ion source. For instance, a capillary (e.g., a pulled quartz capillary) described herein having a nanosized tip may be used to puncture a cell or tissue and withdraw its biomolecular contents. The capillary may then be directly inserted into a vacuum chamber (e.g., having relatively high vacuums, such as those having reduced pressures such as those described herein) and the extracted molecules may be ionized and delivered to a mass spectrometer. Such techniques may be used, for example, to sample relatively small liquid volumes, such as the contents of a single cell. For instance, such a technique may be used for single cell proteomics studies.

As another example, in some embodiments, the ion source described herein is used for propulsion. For instance, forward propulsion of an object may be generated when ions are ejected in the backward direction. In some embodiments, an ion source such as described herein is used in a propulsion system. This can be used, for example, to deliver a high thrust relative to the weight of the ion source due to, e.g., the small size of the ion source. Additionally, in some cases, the propulsion systems can be made compact and consume relatively less fuel compared to conventional propulsion systems.

In addition, some aspects are directed to a mass spectrometer comprising an ion source as described herein. In some cases, the mass spectrometer may include, besides an ion source such as described herein, components such as vacuum chambers (e.g., able to produce any of the reduced pressures described herein), ion optics (e.g., one or more lenses such as Einzel lenses, etc.), mass filters (e.g., quadrupole mass filters, magnetic sector mass filters, etc.), detectors, ion benders, ion traps, or the like. Examples of specific detectors include, but are not limited to, Faraday cups, electron multipliers, dynodes, charge coupled devices (CCDs), CMOS sensors, and phosphor screens, etc. Additional non-limiting examples of mass spectrometers are described in a provisional application filed on Apr. 23, 2021, entitled “Systems and Methods for Single-Ion Mass Spectrometry with Temporal Information,” incorporated herein by reference in its entirety.

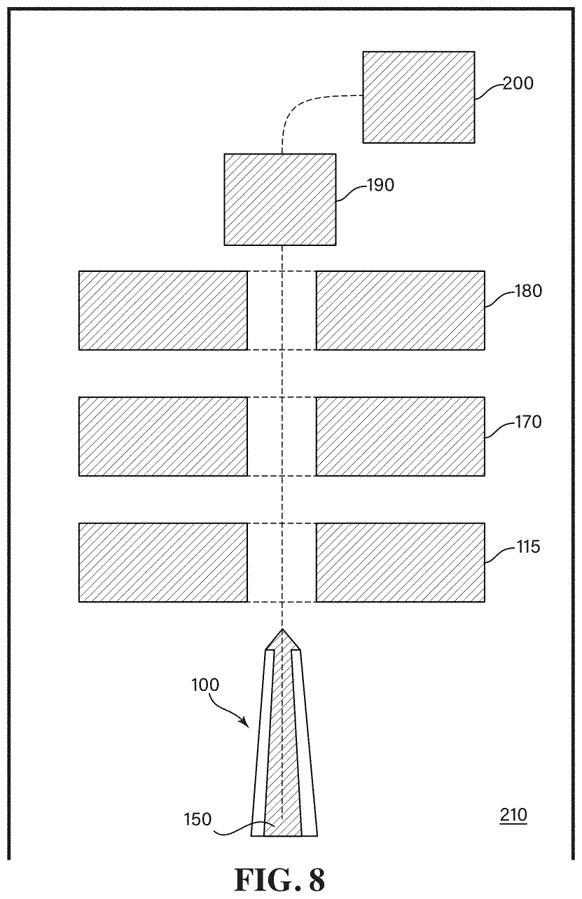

One non-limiting example of an embodiment of a mass spectrometer is now shown in FIG. 8 in a cross-sectional view. In this example, the mass spectrometer has an ion source comprising a capillary 100 and an electrode 115 proximate the capillary 100. The properties of the capillary 100 and the electrode 115 described in this example can be the same as those described elsewhere herein, for example, capillaries with respect to FIGS. 6A-6B and FIGS. 7A-7B. In some embodiments, as a non-limiting example, the capillary may comprise an opening having a cross-sectional dimension of less than 100 nm such that the at least 50% of the molecules exit the opening as ions or ion clusters. In FIG. 8, an electric field proximate the opening of the capillary causes molecules (e.g., ions and ion clusters) within a fluid 150 to exit the fluid into a vacuum chamber 210.

In addition to an ion source, various ion optics can be positioned downstream of the ion source such that the exiting molecules (e.g., ions and ion clusters) can be transported along a path downstream of the ion source in certain cases, e.g., the downstream direction is the direction in which ions or ion clusters travel. Those of ordinary skill in the art will be familiar with various ion optics used in mass spectrometry. In some embodiments, the ion optics comprises one or more Einzel lenses (e.g., a first Einzel lens and a second Einzel lens). For instance, as a non-limiting example, a mass filter 180 is positioned further downstream of the ion optics 170, as shown in FIG. 8. As the ion optics transmit the molecules (e.g., ions or ion clusters) to the mass filter, the mass-to-charge ratios (m/z) of molecules (e.g., ions and ion clusters) may be analyzed by the mass filter. Examples of mass filters include, but are not limited to, quadrupole mass filters, magnetic sector mass filters, etc.

In some embodiments, a detector is positioned further downstream of the mass filter. The detector may be any suitable detector able to detect ions or ion clusters. In some embodiments, ions and ion clusters with mass-to-charge ratios (m/z) within an acceptance window of the mass filter are passed to an ion bender. The ion bender may be configured to deflect the ions and ion clusters leaving the mass filter to a detector. For instance, as a non-limiting example, ions or ion clusters are passed from an ion bender 190 to a detector 200, as shown in FIG. 8. In some embodiments, the detector can be used to determine the ions or ion clusters.

In some embodiments, a mass spectrometer such as described herein may comprise an overall ion transmission (e.g., ratio of ions and ion clusters detected to the ions and ion clusters exiting from the fluid at the opening of the capillary) of greater than 0.01, and in some cases, at transmissions of at least 0.02, at least 0.03, at least 0.05, at least 0.1, at least 0.15, at least 0.2, at least 0.3, at least 0.4, at least 0.5, at least 0.6, at least 0.7, at least 0.75, at least 0.8, etc. In some cases, the overall ion transmission is no more than 1, no more than 0.9, no more than 0.8, no more than 0.75, no more than 0.7, no more than 0.6, no more than 0.5, no more than 0.4, no more than 0.3, no more than 0.2, no more than 0.15, no more than 0.1, no more than 0.05, or no more than 0.02. Combinations of the above-referenced ranges are possible (e.g., at least 0.02 and no more than 0.9, or at least 0.1 and no more than 0.8). Other ranges are also possible.

Certain aspects are directed to sequencing a polymer, such as a biopolymer, using an instrument comprising the ion source, for example, a mass spectrometer such as described herein.

For instance, in some embodiments, a polymer may be the species of interest. The species of interest may be a biopolymer, e.g., a protein or peptide (comprising amino acids), or a nucleic acid sequence (e.g., DNA, RNA, etc.). Other types of biopolymers, such as carbohydrates or polysaccharides, may also be used as a species of interest in some cases. In addition, it should be understood that other types of polymers may also be sequenced in some cases, e.g., artificial or synthetic polymers. Furthermore, analogously, the structures of species of interest that are not polymers may also be determined.

In some cases, for example, the structure, sequence, and/or identity of the species of interest (e.g., a polymer) can be determined by determining the ionized fragments using a detector. For example, the sequence of the species of interest can be detected by monitoring the time of arrival of individual ionized fragments (e.g., ions or ion clusters) at the detector, e.g., that are produced by ionizing the polymer as discussed above, and producing ions or ion clusters. Without wishing to be bound by any theory, it is believed that a species of interest, such as a polymer, may be ionized in substantially linear fashion, e.g., due to the size of the opening of the capillary, and the ions or ion clusters that are produced may then be determined by a detector as discussed herein, e.g., in the order in which the ions or ion clusters are produced from the species of interest. In some embodiments, the capillary comprises a carbon nanotube or a boron nitride nanotube, where the cross-sectional dimension (e.g., inner diameter) of the nanotubes is small enough, e.g., 1 nm to 2 nm, such that a polymer molecule may ionize in a sequential order that reflects the primary structure of the polymer. Of course, larger diameters, or other materials, are also possible in other embodiments, e.g., as discussed herein. It should be noted that in some cases, e.g., when the ions or ion clusters are passed into reduced pressure environments, the detector may be able to determine such ordering at relatively high fidelity, for example due to the relative lack of collisions with gas molecules as the ions or ion clusters pass through to the detector. Accordingly, based on the order at which ions or ion clusters are determined, the structure or sequence of the species of interest can be determined.

U.S. Provisional Patent Application Ser. No. 63/015,407, filed Apr. 24, 2020, entitled “Nanotip Ion Sources and Methods,” by Stein, et al., is incorporated herein by reference in its entirety. In addition, a provisional application filed on Apr. 23, 2021, entitled “Systems and Methods for Single-Ion Mass Spectrometry with Temporal Information,” by Stein, et al., is also incorporated herein by reference in its entirety.

The following examples are intended to illustrate certain embodiments of the present disclosure, but do not exemplify the full scope of the disclosure.

### Example 1

Disclosed in the following examples are certain systems and methods that allowed the measurement of a sample (e.g., amino acids) directly emitted into vacuum (i.e., a reduced pressure environment) via ion evaporation from the surface of a fluid, e.g., an aqueous solution in this example. The systems and methods in these examples were applicable to fluids with relatively low conductivities, e.g., equivalent to about 10 mM NaCl. In some embodiments, the methods also predominantly produced bare ions or ionic clusters with only one or two water molecules. One feature was directed to the nanoscale size (<100 nm) of the ion source. The small size of the capillary tip produced significant field enhancement and restricted the fluid flow rate such that ions from the samples (e.g., amino acids) exited directly into vacuum via ion evaporation rather than from the formation of droplets and a sequence of Coulomb fissions. The small tip opening (sometimes also called a “nanopore”) also prevented the evaporation of significant amounts of solvent into the vacuum chamber while keeping the fluids from freezing, allowed the study of samples such as amino acids in volatile solvents such as water.

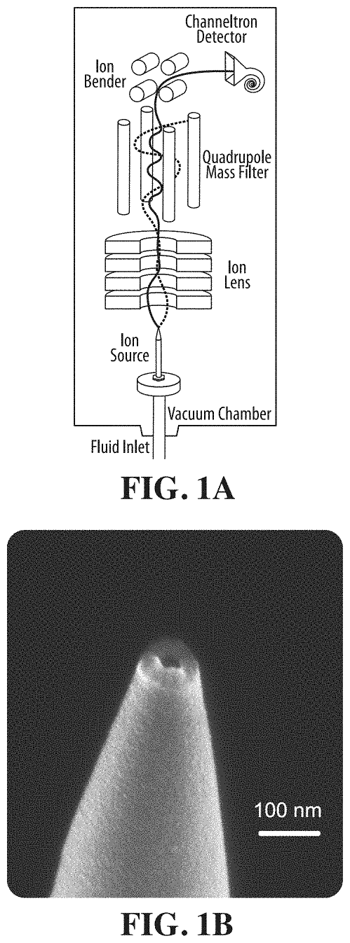

This example illustrates various parts of the mass spectrometer instrument that was used to conduct experiments in accordance with one embodiment. The experiments described in the following examples were all conducted in a custom instrument called a “nanopore mass spectrometer,” illustrated schematically in FIGS. 1A-1B.

One component of the instrument used in this example is an ion source. The ion source includes a capillary with a sub-100 nm inner tip diameter and an annular electrode situated in front of the capillary tip within the system. The capillary was made of quartz in this example, but capillaries could also have been made of borosilicate glass, plastics, metals, ceramics, semiconductors, or other materials in other embodiments. As shown in FIG. 1B, the diameter of the capillary in this example tapered gradually down in size approaching the tip, such that if one approximated the shape of the tip to be a cone, the opening angle of the cone would be in the range of about 1 degree to 5 degrees. The capillary was much longer than it is wide at the tip, giving it an extremely high aspect ratio, typically above 10,000. Of course, as previously discussed, other capillary shapes and/or dimensions can be used in other embodiments.

The electrode was used in this example to induce an electric field at the opening of the tip of the capillary that was high enough to allow ions to exit directly from the meniscus of the fluid created there, at least in part by ion evaporation. The electrode was made of steel in this instrument, but it could have been made from another conducting material. The electrode in this example featured an aperture through which ions could travel, e.g., exiting from the meniscus of the fluid by ion evaporation. In this instrument, the electrode had the shape of a washer, i.e., a circular disk with a circular hole in the middle, although other shapes could have been used. The diameter of the hole in the middle was about 1 cm in this instrument, but that dimension was not critical. For example, it may be at least 10 times larger than the capillary tip diameter, or other dimensions as described herein. The outer diameter of the electrode was about 5 cm, but that dimension was also not critical. It may be larger than the inner hole diameter. The front side of the electrode in this experiment defined a plane, and the tip of the capillary was situated behind that plane at a distance in the range of about 1 mm to 5 mm, with the capillary axis aligned with the axis of the electrode.

A voltage typically in the range 80 V-400 V was applied between the fluid and the electrode to cause ions to exit the fluid. An Ag/AgCl wire in the capillary was used as the counterelectrode. The exiting beam of ions passing through the aperture in the electrode was focused using two Einzel lenses. The ions were analyzed by a quadrupole mass filter in the instrument, but a different type of mass filter, e.g., a magnetic sector, could also have been used.

### Example 2

This example illustrates certain protocols for operating the mass spectrometry instrument described in Example 1. The fluid in the opening was usually biased to a constant potential of 201 V relative to ground by a voltage supply (Keithley 2657A). This was also monitored to measure the current of ions leaving the nanopore. The potential of the electrode was adjusted between −300 V and 300 V to achieve ion emission. A 440 kHz RF oscillator with a mass range of 4000 amu drove the mass filter in this example. The ion current from the source ranged from about 0.001 nA to 0.8 nA. The mass data presented could have been collected for hours, but even after only about 10 minutes, sufficient ions could be collected that a mass spectrum of the sample could be resolved.

### Example 3

This example illustrates a method of capillary preparation for use in an ion source such as described in the previous examples. In this example, nanoscale capillary tips were produced by laser-pulling quartz capillaries. A thin quartz capillary (Sutter Instrument, Ca) with a length of 7.5 cm, an outer diameter of 1.0 mm, and an inner diameter of 0.70 mm with a 0.1 mm filament (ref: QF100-70-7.5) was placed in a laser-based pipette puller (Sutter Instrument, P-2000). The pipette pulling occurred in a one-stage process and different pulling parameters were used to produce capillary tips of various sizes: pulling parameters A (Heat:650; Fil:4, Vel:45, Del:175, Pull:190) were used for producing smaller tips (20-65 nm), and pulling parameters B (Heat:465; Fil:1, Vel:30, Del:145, Pull:175) were used for producing larger (125-300 nm) tips in this example. It should be noted that there were some variations between P-2000 pullers due to local temperature and humidity fluctuations, therefore these pulling parameters only served as examples. After fabrication, the nanoscale capillary tips were carbon coated for imaging using a scanning electron microscope LEO (Zeiss) 1530. The laser-pulled capillaries were imaged and their diameters were measured using the scanning electron microscope (LEO 1530) at 200 k magnification with a voltage of 5 kV to 20 kV. The details of the laser-pulled capillaries are shown in the table below. These laser-pulled capillaries were subsequently used in an ion source of a mass spectrometer to produce mass spectra of various amino acids in Example 6, as described below.

| | Inner Diameter | Outer Diameter | Current Measured |
|---|---|---|---|
| Tip Number | (nm) | (nm) | (nA) |
| 1 | 60 | 115 | 0.1 |
| 2 | 60 | 110 | 0.2 |
| 3 | 20 | 60 | 0.1 |

### Example 4

This example illustrates a method of sample preparation for various amino acid solutions. The single amino acid solutions were prepared by dissolving amino acid powders in ultra-pure water produced from a Q-grad-1 MilliQ system (Millipore) in this example. The pH of the solutions was adjusted by adding glacial acetic acid (Sigma Aldrich, CAS 64-19-7) to reach a pH lower than the isoelectric point (pI) of each amino acid. The pH was measured using an UltraBasic Benchtop Meter from Denver Instrument, and the conductivity was measured by using a Sension+EC71 GLP Conductivity Laboratory Meter (Hach, USA). All amino acids were bought from Sigma Aldrich (97-98% pure). Specific concentrations and properties of each solution are listed in the table below. These various amino acid solutions were prepared for mass spectroscopy studies in the following examples.

| | | | Concen- | | % | Conductivity |
|---|---|---|---|---|---|---|
| Amino Acid | Symbol | pI | tration | pH | Acetic | (S/m) |
| Arginine | Arg | 10.76 | 100 | 8.6 | 0.5 | 0.205 |
| Histidine | His | 7.59 | 100 | 6.22 | 0.3 | 0.247 |
| Lysine | Lys | 9.74 | 100 | 5.75 | 0.6 | 0.486 |
| Glycine | Gly | 5.97 | 100 | 4.00 | 0.1 | 1.956 × 10⁻² |
| Serine | Ser | 5.68 | 100 | 4.08 | 0.1 | 1.563 × 10⁻² |
| Proline | Pro | 6.30 | 100 | 3.84 | 0.1 | 1.396 × 10⁻² |
| Valine | Val | 6.0 | 100 | 4.12 | 0.1 | 1.478 × 10⁻² |
| Threonine | Thr | 5.60 | 100 | 3.94 | 0.1 | 1.403 × 10⁻² |
| Cysteine | Cys | 5.07 | 100 | 3.95 | 0.1 | 1.322 × 10⁻² |
| Leucine | Leu | 5.98 | 100 | 3.86 | 0.1 | 2.10 × 10⁻² |
| Glutamine | Gln | 5.65 | 100 | 4.16 | 0.1 | 1.462 × 10⁻² |
| Methionine | Met | 5.74 | 100 | 3.90 | 0.1 | 1.354 × 10⁻² |
| Alanine | Ala | 6.00 | 100 | 4.10 | 0.1 | 1.740 × 10⁻² |
| Asparagine | Asn | 5.41 | 100 | 3.81 | 0.1 | 1.745 × 10⁻² |
| Phenylalanine | Phe | 5.48 | 100 | 4.10 | 0.1 | 1.080 × 10⁻² |
| Tryptophan | Trp | 5.89 | 50 | 4.00 | 0.1 | 1.519 × 10⁻² |

### Example 5

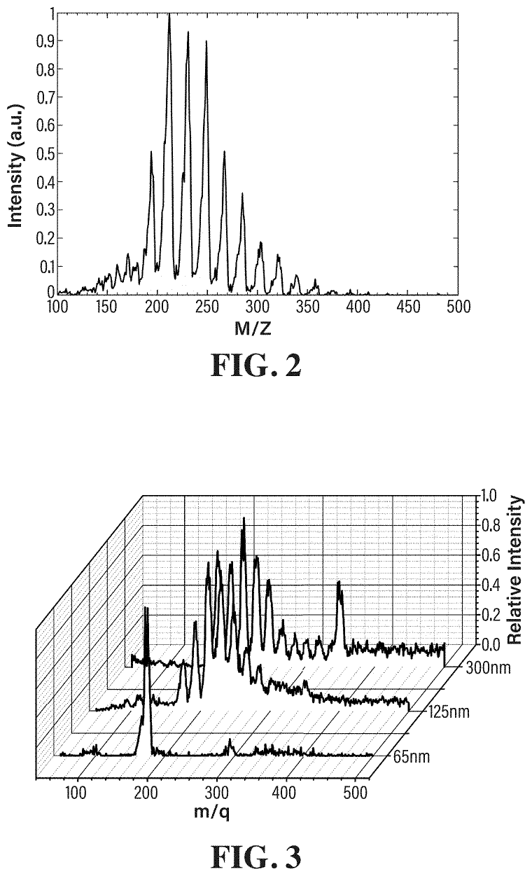

This example illustrates spectra obtained for the amino acid arginine using capillary tips of various sizes. A capillary with a nanoscale tip was used to transfer biomolecules such as amino acids, proteins, and nucleic acids directly from aqueous solution into a high vacuum environment in a charged state. In general, the aqueous solutions measured contained amino acids at concentrations between about 10 mM to 100 mM. To measure the amino acids in a positive ion mode, the pH of each solution was lowered below the isoelectric point of the amino acid of interest. In practice, adding acetic acid in an amount between about 0.1%-1% by volume resulted in solutions with a pH of about 4, which allowed the emission of positively charged ions from solutions of most of the natural amino acids. For some amino acids the pH was as low as 3.8 and for others it was as high as 8.6. Under those chemical conditions and using capillary tips with inner diameters smaller than 300 nm, clean spectra revealing the amino acid of interest were obtained. FIG. 2 shows a mass spectrum obtained from a 100 mM aqueous solution of arginine using a capillary with a tip of about 100 nm. At least 9 mass peaks were visible, with the lightest occurring at m/z=192, corresponding to a singly charged arginine ion (174 amu) complexed with a single water residue (18 amu). The other peaks occurred at increments of 18 m/z, corresponding to the shift induced by an additional water molecule. Thus, the peaks corresponded to a singly charged arginine ion clustered with 2-9 water molecules as shown in FIG. 2.

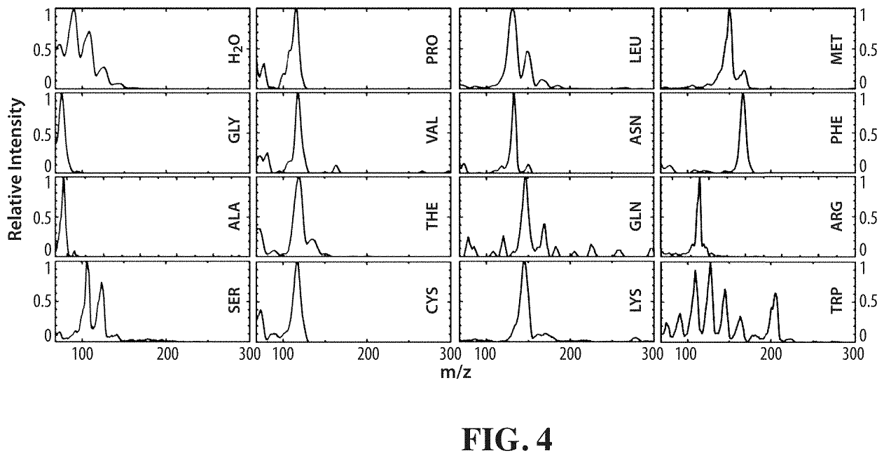

The size of the capillary tip affected the solvation states of the emitted ions. FIG. 3 shows the results of three measurements of a 100 mM aqueous solution of arginine performed using capillary tips with three different inner diameters. The spectrum obtained using a capillary with a 300 nm tip showed 9 peaks separated by 18 m/z, and an additional peak at 349 m/z. The lowest mass peak appeared at 174 m/z, corresponding to a bare arginine ion. The peak at 349 m/z corresponds to a singly charged arginine dimer ion. The spectrum obtained from a 125 nm tip showed 8 peaks, the lowest mass again corresponding to the bare arginine ion. The spectrum obtained from a 65 nm tip revealed just a single peak corresponding to the bare arginine ion. Capillary tips with inner diameters of less than or equal to 300 nm allowed the measurement of amino acid ions exiting from the aqueous solutions. The number of water molecules that accompanied the emitted ions in clusters tended to decrease as the size of the tip decreased. The use of a tip with an inner diameter about 100 nm or smaller was associated with the production of mostly bare ions, i.e., not clustered with water molecules.

### Example 6

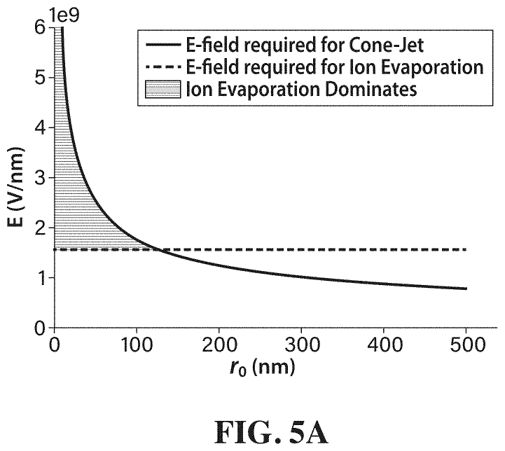

This example illustrates spectra of various amino acids using sub-100 nm capillary tips. This was effective at detecting unsolvated amino acids emitted directly into high vacuum. Using sub-100 nm tips, 16 of the 20 naturally occurring amino acids were measured in a predominantly unsolvated state using nanopore mass spectrometry, as shown in FIG. 4. The results were all obtained using 3 different capillaries with inner diameters ranging from about 20 nm to 60 nm. The extraction voltages applied in these experiments were all in the range of about 260 V-360 V, and the emission currents ranged from about 100 pA-200 pA. In other experiments, similar spectra were obtained using extraction voltages of about 100 V and emission currents of about 10 pA-50 pA (data not shown). The majority of the spectra in FIG. 4—those corresponding to proline, glycine, valine, asparagine, phenylalanine, alanine, threonine, arginine, cysteine, and lysine—all contained just a single dominant mass peak corresponding to the m/z of the amino acid's protonated molar mass, i.e., corresponding to the bare amino acid ion. Four of the amino acid spectra, those of leucine, methionine, threonine, and serine, each showed a smaller second peak, at a mass 18 amu above the main peak. Those secondary peaks corresponded to the amino acid ion clustered with a single water molecule. In the spectrum for tryptophan, seven peaks were evident. The peak at m/z=204 corresponded to the bare tryptophan ion. The other six peaks did not correspond to solvated tryptophan ion clusters, but rather represented the water background, i.e., hydronium clustered with neutral water molecules. The water background was more apparent in the tryptophan spectrum than the others simply because tryptophan ions were emitted at a relatively lower rate. This was in part because tryptophan is poorly soluble in water, so a lower concentration of 50 mM was used for it instead of the 100 mM concentration used for the other amino acids.

### Example 7

This example illustrates experiments performed to analyze the relationship between capillary tip size and ion evaporation. Conventional electrospray ionization experiments operate in the cone-jet regime, in which a fine jet of charged droplets is emitted from the tip of a Taylor cone. For the nanopore mass spectrometer, bare ions and ions clusters were directly extracted from the tip of the capillary via ion evaporation.

Charged moieties can exit or be emitted from a voltage-biased capillary by at least two distinct physical mechanisms, ion evaporation and a liquid jet that breaks up into multiply charged droplets. The use of capillaries with smaller tips favored the ion evaporation mechanism, and the reason related to the characteristic electric fields of the different emission mechanisms. The characteristic field for ion evaporation would need to be lower than that of the formation of a droplet emitting cone-jet. Explained below is why there can be a tip size below which ion evaporation became the main emission mechanism, and that size could be about 100 nm in some cases.

The fields for the formation of a cone-jet and for the onset of ion evaporation are displayed graphically in FIG. 5A, and the cone-jet and ion evaporation mechanisms are illustrated schematically in FIG. 5B and FIG. 5C, respectively. Typical parameters (e.g., field strength at the capillary tip, radius of curvature of the capillary tip, distance of capillary tip from the extractor electrode, the extraction voltage applied) relevant to the experiments conducted with nanoscale capillary tips were used to produce the curves shown in FIG. 5A. The plot showed that below a threshold tip diameter of approximately 120 nm, ion evaporation should be the dominating process for sufficiently strong electric fields. A range of electric field strengths for which only ion evaporation occurred was found and shown in FIG. 5A. A regime in which only ion evaporation occurred is often called the “pure ion evaporation” regime. Furthermore, without wishing to be bound by theory, it is believed that the small size of the capillary tip may restrict the fluid flow rate of the samples, such that ions from the samples (e.g., amino acids) exit directly into vacuum via ion evaporation rather than the formation of charged droplets. The methods described in this example allowed an ion source to deliver biomolecular ions into a mass spectrometer from aqueous solution in a pure ion evaporation regime.

The results presented in this example demonstrated the use of nanoscale ion sources for the analysis of amino acids. The same methods could have been used to analyze larger biomolecules like proteins. These methods had also been successfully applied to analyze glutathione molecules with various post-translational chemical modifications. Again, the absence of a background gas that could cause evaporation of droplets indicated that the measured ions could be emitted in an unhydrated state directly from the aqueous meniscus at the tip, rather than from charged droplets, as described in this example.

In conclusion, the example described here represented a new technique for the soft ionization of biomolecules which allows for various analyses, including single molecule analyses and single molecule protein sequencing.

### Example 8

This example illustrates experiments conducted to highlight the low freezing nature of aqueous samples in capillary tips of small sizes. Typically, introducing a volatile solution directly into high vacuum can create problems for a mass spectrometer. A high rate of evaporation can cause pressure spikes in the instrument, which needs to operate under high vacuum conditions. Also, if the rate of evaporative cooling is high enough, the liquid in the capillary can freeze, which prevents the release of ions and precludes a mass spectrometry measurement.

To suppress these problems, it is possible to use solvents with a low vapor pressure, like formamide or glycerol. But for the analysis of biopolymers, it is desirable to work with aqueous solutions, which are far more volatile. Experiments were conducted to demonstrate that water-filled capillaries with diameters larger than about 500 nm tended to freeze, whereas capillaries with smaller diameters tended not to in this example. For certain experiments conducted using water-filled capillaries with diameters larger than about 500 nm, no ion emission currents were produced, and no mass spectra were obtained in those experiments. The reason small tips avoided the problem of freezing could be understood based on a scaling argument: The rate of heat loss due to evaporation is proportional to the surface area of the liquid-vacuum interface at the end of the tip, whereas the rate at which conduction delivered heat to the tip is approximately proportional to the tip diameter, therefore the degree of cooling achieved at the tip is smaller for smaller capillary tips. In other words, it could be assumed that the heat flowing out of the solvent due to evaporation was proportional to the exposed surface area at the end of the tip. This meant that the temperature at the end of the tip was lower than the ambient temperature by an amount proportional to the final radius of the tip. Therefore, with sufficiently small capillaries, the temperature at the tip should have been close to room temperature, while for larger capillaries the temperature may have been significantly lower, leading to freezing of the solvent in the capillary tip. This result was borne out by the successful experiments conducted with aqueous solutions of amino acids using capillaries with nanoscale openings as described in the aforementioned examples, as these experiments would fail if the solutions were freezing.

### Example 9

This example illustrates experiments conducted to highlight the high ion transmission efficiency achieved using nanocapillary ion sources. Conventional electrospray ionization MS relies on a background gas to aid droplet evaporation in order to produce ions. This necessitates the use of a thin transfer capillary to bring ions from the atmospheric pressure region housing the ion source to the high vacuum region housing the mass analyzer. The majority of the ions emitted from the source are typically intercepted by the walls of the transfer capillary, causing a low ion transmission efficiency and limiting the sensitivity of the technique. Using the nanocapillary ion source, ions are emitted directly into high vacuum, obviating the need for a transfer capillary between high pressure and low pressure regions.

The ion transmission efficiency can be measured as the ratio of current leaving the nanotip and the current striking the Faraday plate. Using sub-100 nm tips, a solution of 1 M NaI in formamide was emitted into a vacuum chamber containing a set of ion optics and a Faraday plate positioned past the ion optics. Currents and efficiency were measured over a short period during the experiment. Emission currents ranging from 2 nA and 10 nA were measured, and the current detected striking the Faraday plate was less by about 25%, resulting in an ion transmission efficiency of around 75%. In other experiments, the same type of measurement was carried out, but with a magnetic sector mass analyzer installed in the vacuum chamber. The magnetic sector would deflect any ions away from the Faraday plate, but allow droplets to pass through with minimal deflection and strike the Faraday plate, producing a detectable current. In this configuration, an emission current of 20 nA was measured but only 10 pA were detected at the Faraday plate, indicating that the vast majority of the emitted current was in the form of ions.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An ion source, comprising:
a capillary defining an opening having a cross-sectional dimension of less than 100 nm; and
an electrode positioned proximate the opening of the capillary in a downstream direction.

2. The ion source of claim 1, wherein the opening of the capillary has a cross-sectional dimension of less than 65 nm.

3-5. (canceled)

6. The ion source of claim 1, wherein the capillary is tapered at the opening.

7-16. (canceled)

17. The ion source of claim 1, wherein the capillary has an aspect ratio of length to cross-sectional dimension of greater than or equal to 100.

18-19. (canceled)

20. The ion source of claim 1, wherein the capillary has a cross-sectional dimension of less than 100 nm.

21-24. (canceled)

25. The ion source of claim 22, wherein the center opening of the electrode is larger than the opening of the capillary.

26-28. (canceled)

29. The ion source of claim 1, wherein the electrode is annular.

30. The ion source of claim 1, wherein the electrode has a cross-sectional dimension of less than 5 cm.

31. The ion source of claim 1, wherein the electrode is positioned within 10 mm of the opening of the capillary.

32-33. (canceled)

34. The ion source of claim 1, wherein the electrode is positioned around the capillary.

35. The ion source of claim 1, wherein the electrode is positioned in front of the opening of the capillary.

36. (canceled)

37. The ion source of claim 1, wherein the electrode and the capillary has an interior connected to a voltage source.

38-40. (canceled)

41. The ion source of claim 37, wherein the voltage source is capable of producing an electric field between the electrode and the capillary having a maximum of less than or equal to 4 V/nm.

42-45. (canceled)

46. A mass spectrometer, comprising:
the ion source of claim 1;
ion optics downstream of the ion source;
a mass filter downstream of the ion optics; and
a detector downstream of the mass filter.

47-74. (canceled)

75. A method, comprising:
passing a fluid into a capillary defining an opening; and
applying an electric field at least sufficient to cause molecules within the fluid to exit the fluid, wherein the opening is sized to cause at least 50% of the molecules to exit as ions or ion clusters.

76. The method of claim 75, further comprising determining the ions or ion clusters.

77-80. (canceled)

81. The method of claim 76, wherein the ion clusters contain an average of no more than 7 molecules of solvent.

82-84. (canceled)

85. The method of claim 75, wherein the opening of the capillary is sized such that, when the electric field is applied, at least 50% of the exiting species exit the charged meniscus via ion evaporation.

86-89. (canceled)

90. The method of claim 75, further comprising sequencing the ions or ion clusters to determine the molecule.

91-96. (canceled)

97. The method of any ono of claim 75, wherein the molecules exiting as ions or ion clusters exit at an overall ion transmission efficiency of greater than 0.1.

98-107. (canceled)
